(19) United States
(12) Patent Application Publication
(10) Pub. No.: US 2025/0255959 A1
(43) Pub. Date: Aug. 14, 2025

(54) A SYSTEM FOR DRUG-INDUCIBLE EXPRESSION OF A POLYNUCLEOTIDE

(71) Applicant: Miltenyi Biotec B.V. & Co. KG, Bergisch Gladbach (DE)

(72) Inventors: Bettina Kotter, Bergisch Gladbach (DE); Joerg Mittelstaet, Bergisch Gladbach (DE); Andrew Kaiser, Bergisch Gladbach (DE); Andre Roy, Bergisch Gladbach (DE); Winfried Krueger, Bergisch Gladbach (DE)

(21) Appl. No.: 18/851,192

(22) PCT Filed: Mar. 30, 2023
(86) PCT No.: PCT/EP2023/058248

§ 371 Date: Sep. 26, 2024

(30) Foreign Application Priority Data
Apr. 1, 2022 (EP) 22166403.0

(51) Int. Cl. A61K 40/31; A61K 40/11; A61K 40/42; A61P 35/00; C07K 14/725; C07K 16/28; C12N 15/86

(52) U.S. Cl. CPC A61K 40/31; A61K 40/11; A61K 40/4221; A61P 35/00; C07K 14/7051; C07K 16/2887; C12N 15/86; C12N 2740/15043; C12N 2830/002

(57) ABSTRACT

The present invention provides a system for drug-inducible expression of a polynucleotide comprising a) a first nucleic acid sequence comprising a first promoter inducible by said drug, wherein the first promoter is operably linked to said polynucleotide, wherein said first promotor comprises a binding site for a DNA binding domain, wherein said binding site comprises at least one responsive element that is recognized by said DNA binding domain (DBD), and b) a second nucleic acid sequence comprising a second promoter, wherein the second promoter is operably linked to a nucleic acid sequence encoding a synthetic transcription factor, wherein said synthetic transcription factor comprises i) an activation domain (AD), wherein said AD comprises the p65 activation domain of the human transcription factor NFκB or a functional variant thereof, ii) said DNA binding domain (DBD), wherein said DBD comprises or consists of 3 zinc finger domains, iii) a ligand-binding domain (LBD), wherein said LBD is a modified human estrogen receptor which is able to bind said drug, and wherein said ligand-binding domain (LBD) is positioned at the C-terminus of said synthetic transcription factor, and c) said drug, wherein said drug is tamoxifen or a metabolite of tamoxifen.

### FIELD OF THE INVENTION

The invention relates to a system for drug-inducible expression of a polynucleotide in human immune cells, in particular to a system for drug-inducible expression using a synthetic transcription factor having a defined order of the domains.

### BACKGROUND OF THE INVENTION

Synthetic transcription factors composed of a DNA-binding domain (DBD), a drug-responsive ligand-binding domain (LBD) and an activation domain (AD) represent an attractive switch module for transcriptional regulation of transgenes of interest.

Drug-dependent transcription factors based on a zinc-finger derived DBD, murine estrogen receptor (ER)-based LBD, and VP16 or VP64 as AD (both viral origin) have been described 15 earlier (Beerli et al., 2000, J. Biol. Chem. 275, 32617-32627). While human zinc finger domains are abundantly known for DBDs, use of human ER-derived LBDs has been shown e.g. for Cre-recombinase (Feil et al., 1997, Biochem. Biophys. Res. Commun. 237, 752-757), and human p65 AD in combination with zinc finger based DBD have been described for transcriptional regulation (Liu et al., 2001, J. Biol. Chem. 276, 11323-11334).

Adoptive transfer of CAR immune cells such as CAR T cells has demonstrated remarkable success in treatment of hematological malignancies. However, lack of control of CAR immune cell function and consequent excessive inflammation in patients can result in severe side effects, especially when targeting tumor-associated rather than tumor-specific antigens.

Thus, temporal, tunable and spatial control of CAR activity is of major importance. Furthermore, other genes of therapeutic relevance have been expressed in CAR immune cells like cytokines, checkpoint inhibitors, and others, tight control of the expression and activity of these genes, e.g. by an orally available drug, would offer the potential to increase therapeutic efficacy and limit side effects, several approaches have been taken to modulate CAR signaling and thus control activity (e.g, split CAR). In contrast, CAR expression was so far only regulated by employing the Tet-on System (Sakemura, R. et al. 2016, Cancer Immunol. Res. 4, 658-68; Gu, X.et al, 2018, Int. J. Mol. Sci. 19, 1-12; Drent, E. et al. 2018, PLoS One 13, e0197349). While effective induction rates could be obtained in presence of doxycycline, basal expression activity in absence of the drug still represents an important limitation of the system. Furthermore, the Tet-on System is based on protein sequences derived from bacteria and virus, and therefore holds a significant risk for immunogenicity, raising safety concerns for potential clinical applications.

Therefore, there is a need in the art for an improved or alternative drug-inducible system for expression control of therapeutically active genes for e.g. for adoptively transferred immune cells, preferentially human immune cells, with low potential for immunogenicity.

### BRIEF DESCRIPTION OF THE INVENTION

The inventors surprisingly found that a drug-inducible synthetic transcription factor based on the human ER-derived ligand binding domain, the human p65 activation domain of NFκB and a zinc-finger based DNA binding domain having 3 zinc finger domains are well suited for effective induction in the presence of a drug, that is tamoxifen or a derivative thereof, and that show low background expression in the absence of the drug only if the domains of the synthetic transcription factor are arranged in a specific order.

In contrast to well-known drug-inducible expression systems such as the Tet-on System that are based at least in part on protein sequences derived from species other than human, e.g. bacteria and virus, the drug-inducible expression system as disclosed herein has a reduced (or low) or diminished risk for immunogenicity in humans, when said expression system is used in human cells for clinical or therapeutic applications.

In particular, the invention is about a drug-inducible transcription factor, which is responsive to tamoxifen or tamoxifen metabolites and features a modified human ER as LBD, a DNA-binding domain comprising or consisting of 3 zinc-finger domains and an AD derived from human activation domain of NFκB p65, wherein C-terminal positioning of the ligand-binding domain (LBD) is required to prevent background expression in the non-induced state while enabling transcriptional activity upon addition of the drug.

Therefore, in a preferred embodiment of the invention said positioning of individual domains in the drug-inducible transcription factor as disclosed herein is used as a drug-inducible expression system in human cells, preferentially in human immune cells such as primary T cells for expressing a chimeric antigen receptor (CAR).

### BRIEF DESCRIPTION OF THE DRAWINGS

FIG. 1: Schematic presentation of a synthetic transcription factor and the switch concept. (A) Overview of the inducible expression system with the possibility to control any transgene of interest. The synthetic transcription factor is composed of a DNA-binding domain, a ligand-inducible domain and an activation domain. Transcription of the transgene is initiated upon binding of the synthetic transcription factor to its response elements upstream of the minimal promoter. (B) Overview of anti-CD20 CAR induction in T cells. The “on” and “off”-status of inducible anti-CD20 CAR T cells can be controlled by the administration of the inducer drug.

FIG. 2: Induction of anti-CD20 CAR expression in primary T cells. T cells were transduced with the inducible switch system comprising a synthetic transcription factor composed of the N1 zinc finger protein, the ligand-binding domain of the human estrogen receptor and an activation domain derived from NFκB p65 in different orientations of the 10 individual domains as well as with the constitutively expressed anti-CD20 CAR (“direct CAR”). T cells were cultured in the presence of 0-250 nM 4-OHT for 40 h and analyzed for anti-CD20 CAR expression by flow cytometry using anti-CD20 CAR detection reagent-PE. Frequency of anti-CD20 CAR+inducible T cells (left graph) and mean fluorescence intensity (MFI) of anti-CD20 CAR expression (right graph) was determined and normalized to the expression of the 15 transduction marker LNGFR

FIG. 3: Cytotoxic activity of inducible anti-CD20 CAR T cells in co-cultures with GFP+CD20+Mel526. Inducible anti-CD20 CAR T cells were co-cultured with GFP+CD20+526-Mel at an E:T ratio of 1:1 in the absence or presence of 100 nM 4-OHT added at the start of the 20 assay. The growth of tumor cells was monitored in 2 h intervals over a period of 100 h using a live-cell imaging device (IncuCyte®). Untransduced T cells served as a negative control, while the conventional constitutively expressed anti-CD20 CAR (“direct CAR”) represented the positive control.

FIG. 4: Cytokine secretion of inducible anti-CD20 CAR T cells in co-cultures with GFP+CD20+Mel526. Inducible anti-CD20 CAR T cells were co-cultured with GFP+CD20+526-Mel at an E:T ratio of 1:1 in the absence or presence of 100 nM 4-OHT added at the start of the assay. The concentration of human IFN-7 (left graph) and I1L-2 (right graph) were measured in the supernatants of co-cultures 40 h post assay initiation using the MACSPlex Cytokine kit.

FIG. 5: Cytotoxic activity of inducible anti-CD20 CAR T cells in vivo. NSG™ mice were inoculated with 4×10⁵ RajiffLuc cells via tail-vein injection on day -7. On day 0, following randomization, mice were treated with untransduced, inducible or conventional constitutively expressed (“direct”) anti-CD20 CAR T cells. Tamoxifen was administered daily by i.p. injections to the indicated groups starting on day -1. Tumor burden was regularly determined by in vivo BLI and expressed in photon flux [photons/see] over time for individual mice.

FIG. 6: Positioning of the activation domain within the synthetic transcription factor determines background expression in the non-induced state and the transcriptional output. T cells were transduced with synthetic transcription factors comprising a zinc finger protein (N1), the ligand binding domain of the estrogen receptor (ER), and an activation domain (p65) in distinct arrangements. Expression of an anti-CD20 CAR was induced by the addition of 0-250 nM 4-OHT to the cell culture. Anti-CD20 CAR expression was analyzed 40 h after induction by flow cytometry and normalized to the expression of the transduction marker LNGFR. Data for frequency of anti-CD20CAR+ T cells (left graph) and MFI of anti-CD20 CAR+ T cells (right graph) were plotted.

FIG. 7: The selection of the activation domain within the synthetic transcription factor determines background expression in the non-induced state and the transcriptional output. T cells were transduced with synthetic transcription factors comprising a zinc finger protein (N1), the ligand binding domain of the estrogen receptor (ER), and distinct activation domains (NFκB p65 (aa361-551), NFκB p65 (aa288-548), a fusion protein of NFκB p65 and HSF-1, or VP64) in distinct arrangements. Expression of an anti-CD20 CAR was induced by the addition of 0-250 nM 4-OHT to the cell culture. Anti-CD20 CAR expression was analyzed 40 h after induction by flow cytometry and normalized to the expression of the transduction marker LNGFR. Data for frequency of anti-CD20CAR+ T cells (left graph) and MFI of anti-CD20 CAR+ T cells (right graph) were plotted.

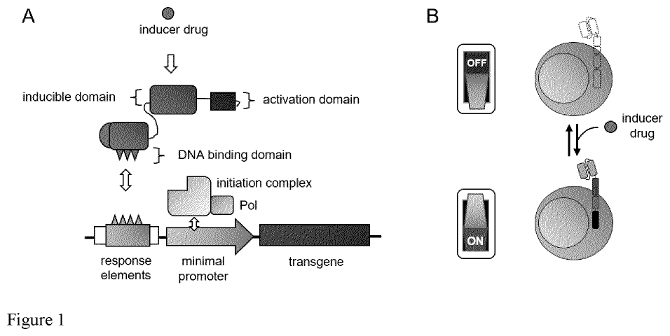

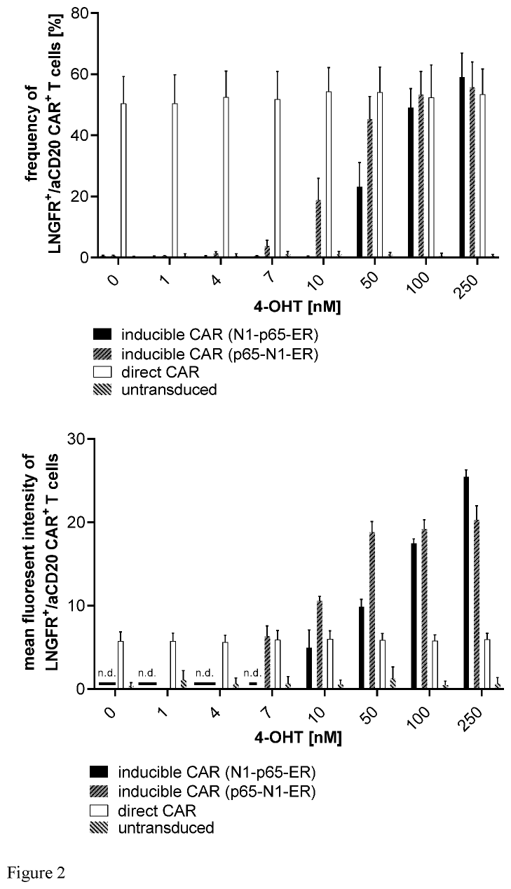

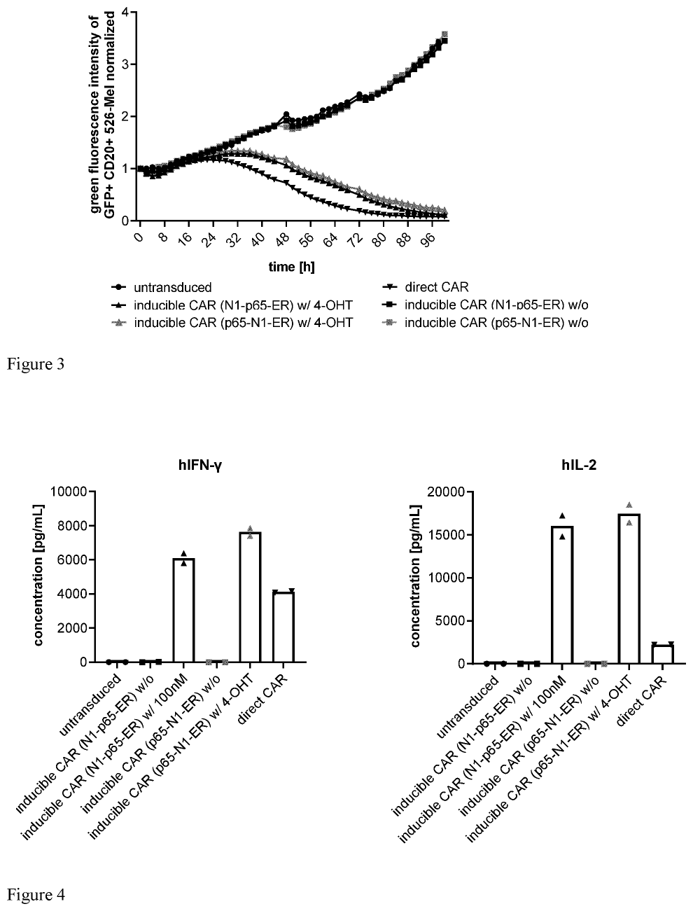

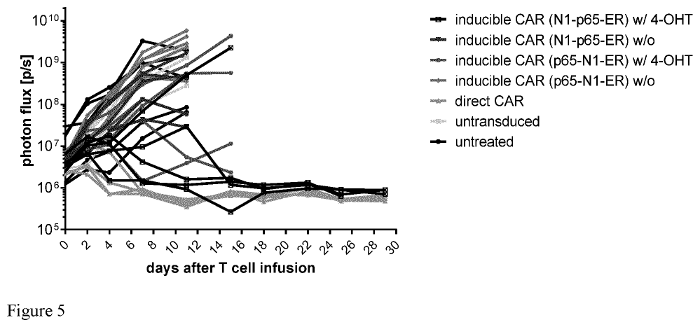

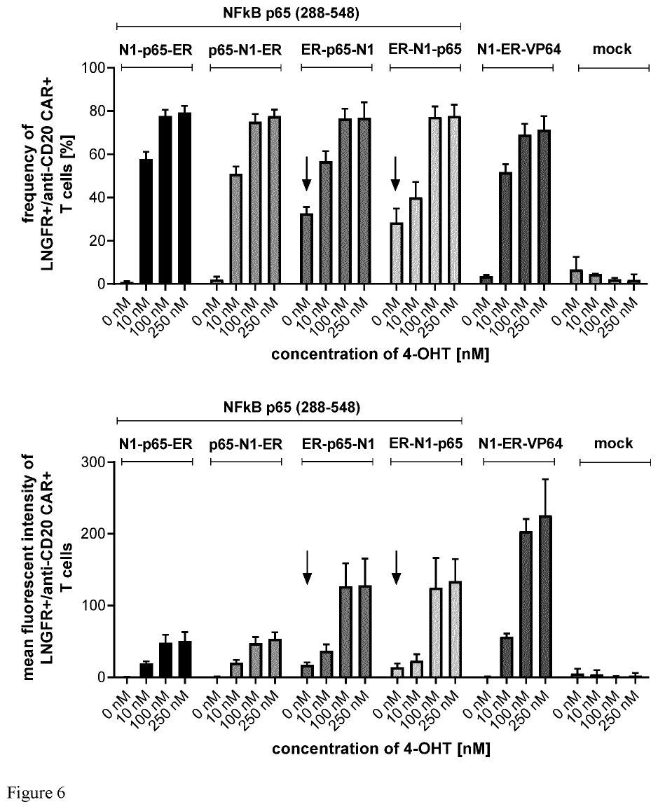

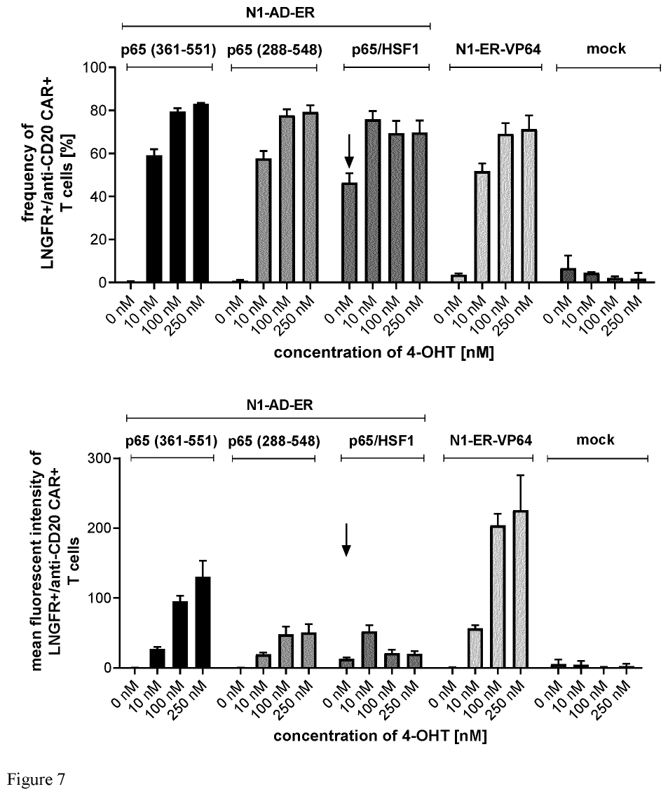

### DETAILED DESCRIPTION OF THE INVENTION

In a first aspect the present invention provides a system for drug-inducible expression of a polynucleotide (in a human immune cell) comprising
a) a first nucleic acid sequence comprising a first promoter inducible by said drug, wherein the first promoter is operably linked to said polynucleotide, wherein said first promotor comprises a binding site for a DNA binding domain, wherein said binding site comprises at least one responsive element that is recognized (bound) by said a DNA binding domain (DBD), and
b) a second nucleic acid sequence comprising a second promoter, wherein the second promoter is operably linked to a nucleic acid sequence encoding a synthetic transcription factor, wherein said synthetic transcription factor comprises
i) an activation domain (AD), wherein said AD comprises the p65 activation domain of the human transcription factor NFκB or a functional variant thereof
ii) said DNA binding domain (DBD), wherein said DBD comprises or consists of 3 zinc finger domains
iii) a ligand-binding domain (LBD), wherein said LBD is a modified human estrogen receptor which is able to bind said drug, wherein said drug is tamoxifen or a metabolite of tamoxifen, c) said drug.

Said DNA binding domain (DBD) may be a polypeptide that has 3 zinc-finger domains. Each of said zinc-finger domains may comprise a recognition sequence determining the specificity of the domain to the responsive element of the DNA binding site.

Said recognition sequence may be e.g. SEQ ID NO:1 (recognition sequence position -2 to 6 (relative to the start of the alpha-helix) of a finger that recognizes the target site GAA) and/or SEQ ID NO:2 (recognition sequence position -2 to 6 (relative to the start of the alpha-helix) of a finger that recognizes the target site GTA) and/or SEQ ID NO:3 (recognition sequence position -2 to 6 (relative to the start of the alpha-helix) of a finger that recognizes the target site GGG).

A zinc finger domain recognizes always 3 bp, the target site. The amino acids at position -1, 2, 3, 6 with respect to the start of the α-helix of the zinc finger domain are responsible for the DNA recognition.

A consensus zinc-finger-framework sequence derived from native and mutant versions of SpI zinc fingers may be SEQ ID NO: 4.

Said zinc-finger protein may be the N1 zinc-finger protein (N1 ZFP or N1).

Said N1 may comprise the sequence of SEQ ID NO:5.

The corresponding responsive element for N1 may be SEQ ID NO:6.

Said binding site may comprise 1, 2, 3, 4, 5, 6, 7, 8, 9 or more responsive element(s) that may be recognized (bound) by said a DNA binding domain (DBD).

Said binding site may comprise five responsive elements, wherein each responsive element may comprise two direct repeats of the recognition motif SEQ ID NO:7 spaced by 3 or 4 base-pairs (bps), if said zinc-finger protein is N1 ZFP.

Said polynucleotide may be a polynucleotide coding for a peptide or polypeptide or protein.

Said protein may be e.g. a chimeric antigen receptor (CAR), a cytokine or an antibody or a fragment thereof such as a Fab.

Said polynucleotide may be a polynucleotide coding for a therapeutically active protein.

Said polynucleotide may be a polynucleotide miRNA, siRNA or shRNA.

Said first promoter may be an inducible promoter, wherein said first promotor may be inducible by the drug as disclosed herein and wherein said first promoter may comprise a binding site for a DNA binding domain, wherein said binding site comprises at least one responsive element that is recognized (bound) by the DNA binding domain (DBD).

Said second promoter may be a constitutive promotor.

Said second promoter may be for example EF-1 alpha promoter or any other constitutive promoter that drives constitutive expression in immune cells such as MSCV, PGK-1, UBC, CMV, CAGG, SV40 or pan-hematopoietic promoter, such as vav.

Said second promoter may be EF1α.

Said drug may be tamoxifen or a metabolite of tamoxifen, or an analog of tamoxifen.

Said metabolite of tamoxifen, or an analog of tamoxifen may be 4-hydroxytamoxifen (4-OHT) or endoxifen.

Said p65 activation domain of the human transcription factor NFκB may be e.g. the complete sequence of SEQ ID NO:8, or aa361 to aa551 of SEQ ID NO:8, or aa288 to aa548 of SEQ ID NO:8, or aa 360 to aa551 of SEQ ID NO:8.

Said modified human estrogen receptor may comprise a modification that may result in the loss of responsiveness to the natural ligand, i.e. human estrogen, but not to a synthetic ligand, i.e. the drug such a metabolite of tamoxifen such as 4-hydroxytamoxifen or endoxifen. Said modified human estrogen receptor, wherein said modified human estrogen receptor may comprise the amino acid substitutions G400V, M543A, L544A or G400V, L540A, M543A as compared to the wild type amino acid sequence of human estrogen receptor.

The unmodified human ER part usable for the synthetic transcription factor as disclosed herein may comprise SEQ ID NO:9 (human ER aa282 to aa595).

Said modified human estrogen receptor, wherein said modified human estrogen receptor may comprise SEQ ID NO:10 (human ER (G400V, L540A, M543A)) or SEQ ID NO:11 (human ER (G400V, M543A, L544A)) or SEQ ID NO:12 (human ER (G400V/L539A/L540A)).

The system as disclosed herein, wherein said polynucleotide encodes a chimeric antigen receptor (CAR) or an exogenous T cell receptor (TCR).

Said CAR may comprise
i) an antigen binding domain specific for an antigen expressed on the surface of a target cell or specific for a soluble antigen or specific for a tag of a tagged polypeptide, wherein said tagged polypeptide may be specific for an antigen on the surface of a target cell
ii) a transmembrane domain
iii) an intracellular signaling domain.

The system as disclosed herein, wherein said polynucleotide encodes a chimeric antigen receptor (CAR), wherein said CAR is expressed in a human immune cell such as a T cell such as a primary T cell or an NK cell.

The system as disclosed herein, wherein said first nucleic acid sequence and said second nucleic acid sequence are on different nucleic acid sequences or wherein said first nucleic acid sequence and said second nucleic acid sequence are on one nucleic acid sequence.

Said one nucleic acid sequence may be a vector such as a retroviral vector. Said retroviral vector may be a lentiviral vector.

Said vector such as a retroviral vector or lentiviral vector may comprise said first nucleic acid sequence in 3′ to 5′ orientation (as compared to said second nucleic acid sequence). This is for avoidance of readthrough and resulting background transcription in the absence of said drug.

In a further aspect the present invention provides a composition comprising
A) a human immune cell comprising
a) a first nucleic acid sequence comprising a first promoter inducible by a drug, wherein the first promoter is operably linked to said polynucleotide, wherein said first promotor comprises a binding site for a DNA binding domain, wherein said binding site comprises at least one responsive element that is recognized (bound) by the DNA binding domain (DBD), and
b) a second nucleic acid sequence comprising a second promoter, wherein the second promoter is operably linked to a nucleic acid sequence encoding a synthetic transcription factor, wherein said synthetic transcription factor comprises
i) an activation domain (AD), wherein said AD comprises the p65 activation domain of the human transcription factor NFκB or a functional variant thereof
ii) a DNA binding domain (DBD), wherein said DBD comprises or consists of 3 zinc finger domains
iii) a ligand-binding domain (LBD), wherein said LBD is a modified human estrogen receptor which is able to bind said drug, and wherein said ligand-binding domain (LBD) is positioned at the C-terminus of said synthetic transcription factor,
B) said drug wherein said drug is tamoxifen or a metabolite of tamoxifen.

Said human immune cell may be a T cell or an NK cell.

Said polynucleotide may be a polynucleotide coding for a CAR. Said CAR may be specific for a tumor antigen or a tumor associated antigen (TAA), e.g. expressed on the cell surface of a tumor (target) cell, or may be a soluble antigen.

In another aspect the present invention provides a composition for use in treatment of a disease (in a subject) comprising
A) a human immune cell comprising
a) a first nucleic acid sequence comprising a first promoter inducible by drug, wherein the first promoter is operably linked to a polynucleotide (to be expressed), wherein said first promotor comprises a binding site for a DNA binding domain, wherein said binding site comprises at least one responsive element that is recognized (bound) by the DNA binding domain (DBD), and
b) a second nucleic acid sequence comprising a second promoter, wherein the second promoter is operably linked to a nucleic acid sequence encoding a synthetic transcription factor, wherein said synthetic transcription factor comprises
i) an activation domain (AD), wherein said AD comprises the p65 activation domain of the human transcription factor NFκB or a functional variant thereof
ii) a DNA binding domain (DBD), wherein said DBD comprises or consists of at least 3 zinc finger domains
iii) a ligand-binding domain (LBD), wherein said LBD is a modified human estrogen receptor which is able to bind said drug, and wherein said ligand-binding domain (LBD) is positioned at the C-terminus of said synthetic transcription factor,
B) said drug wherein said drug is tamoxifen or a metabolite of tamoxifen.

Said disease may be a cancer, an autoimmune disease, or an infectious disease.

In another aspect the present invention provides a combination of pharmaceutical compositions comprising a first composition and a second composition, the first composition comprising a human immune cell comprising
a) a first nucleic acid sequence comprising a first promoter inducible by drug, wherein the first promoter is operably linked to a polynucleotide (to be expressed), wherein said first promotor comprises a binding site for a DNA binding domain, wherein said binding site comprises at least one responsive element that is recognized (bound) by the DNA binding domain (DBD), and
b) a second nucleic acid sequence comprising a second promoter, wherein the second promoter is operably linked to a nucleic acid sequence encoding a synthetic transcription factor, wherein said synthetic transcription factor comprises
i) an activation domain (AD), wherein said AD comprises the p65 activation domain of the human transcription factor NFκB or a functional variant thereof
ii) a DNA binding domain (DBD), wherein said DBD comprises or consists of 3 zinc finger domains
iii) a ligand-binding domain (LBD), wherein said LBD is a modified human estrogen receptor which is able to bind said drug, and wherein said ligand-binding domain (LBD) is positioned at the C-terminus of said synthetic transcription factor, and optionally
c) a pharmaceutically acceptable carrier, and
B) said drug, wherein said drug is tamoxifen or a metabolite of tamoxifen, and optionally a pharmaceutically acceptable carrier.

Pharmaceutically acceptable carriers, diluents or excipients may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In a further aspect the present invention provides an in-vivo method for treating a disease in a subject, the method comprising
A) administering to said subject a human immune cell comprising
a) a first nucleic acid sequence comprising a first promoter inducible by a drug, wherein the first promoter is operably linked to a polynucleotide (to be expressed), wherein said first promotor comprises a binding site for a DNA binding domain, wherein said binding site comprises at least one responsive element that is recognized (bound) by the DNA binding domain (DBD), and
b) a second nucleic acid sequence comprising a second promoter, wherein the second promoter is operably linked to a nucleic acid sequence encoding a synthetic transcription factor, wherein said synthetic transcription factor comprises
i) an activation domain (AD), wherein said AD comprises the p65 activation domain of the human transcription factor NFκB or a functional variant thereof
ii) a DNA binding domain (DBD), wherein said DBD comprises or consists of 3 zinc finger binding domains, and
iii) ligand-binding domain (LBD), wherein said LBD is a mutated human estrogen receptor which is able to bind said drug, and wherein said ligand-binding domain (LBD) is positioned at the C-terminus of said synthetic transcription factor,
b) administering to said subject said drug, wherein said drug is tamoxifen or a metabolite of tamoxifen.

In another aspect the present invention provides a kit for drug-inducible expression of a polynucleotide (in a human immune cell) comprising
a) a first nucleic acid sequence comprising a first promoter inducible by said drug, wherein the first promoter is operably linked to said polynucleotide, wherein said first promotor comprises a binding site for a DNA binding domain, wherein said binding site comprises one at least one responsive element that is recognized (bound) by said a DNA binding domain (DBD), and
b) a second nucleic acid sequence comprising a second promoter, wherein the second promoter is operably linked to a nucleic acid sequence encoding a synthetic transcription factor, wherein said synthetic transcription factor comprises
i) an activation domain (AD), wherein said AD comprises the p65 activation domain of the human transcription factor NFκB or a functional variant thereof
ii) said DNA binding domain (DBD), wherein said DBD comprises or consists of 3 zinc finger domains
iii) a ligand-binding domain (LBD), wherein said LBD is a modified human estrogen receptor which is able to bind said drug, and wherein said ligand-binding domain (LBD) is positioned at the C-terminus of said synthetic transcription factor,
c) said drug, wherein said drug is tamoxifen or a metabolite of tamoxifen.

All definitions, characteristics and embodiments defined herein with regard to the first aspect of the invention as disclosed herein also apply mutatis mutandis in the context of the other aspects of the invention as disclosed herein.

### Definitions

Unless defined otherwise, technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not. The term, system” in the context of a system for drug-inducible expression of a polypeptide (in cells) may refer to a combination of nucleic acids (nucleic acid sequences) present e.g. in a human immune cell and the drug as disclosed herein.

In general, a CAR may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In some embodiments of the invention the antigen binding domain of a CAR binds a tag or hapten that is coupled to a polypeptide (“haptenylated” or “tagged” polypeptide), wherein the polypeptide may bind to a disease-associated antigen such as a tumor associated antigen (TAA) that may be expressed on the surface of a cancer cell.

Such a CAR may be referred to as “anti-tag” CAR or “adapterCAR” or “universal CAR” as disclosed e.g. in U.S. Pat. No. 9,233,125B2.

The haptens or tags may be coupled directly or indirectly to a polypeptide (the tagged polypeptide), wherein the polypeptide may bind to said disease associated antigen expressed on the (cell) surface of a target. The tag may be e.g. dextran or a hapten such as biotin or fluorescein isothiocyanate (FITC) or phycoerythrin (PE) or thiamin, but the tag may also be a peptide sequence e.g. chemically or recombinantly coupled to the polypeptide part of the tagged polypeptide. The tag may also be streptavidin. The tag portion of the tagged polypeptide is only constrained by being a molecule that can be recognized and specifically bound by the antigen binding domain specific for the tag of the CAR. For example, when the tag is FITC (Fluorescein isothiocyanate), the tag-binding domain may constitute an anti-FITC scFv.

Alternatively, when the tag is biotin or PE (phycoerythrin), the tag-binding domain may constitute an anti-biotin scFv or an anti-PE scFv, respectively.

A “signal peptide” refers to a peptide sequence that directs the transport and localization of the protein within a cell, e.g. to a certain cell organelle (such as the endoplasmic reticulum) and/or the cell surface.

Generally, an “antigen binding domain” refers to the region of the CAR that specifically binds to an antigen, e.g. to a tumor associated antigen (TAA) or tumor specific antigen (TSA). The CARs of the invention may comprise one or more antigen binding domains (e.g. a tandem CAR).

Generally, the targeting regions on the CAR are extracellular. The antigen binding domain may comprise an antibody or an antigen binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G4/S)₃-linker”.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

“Spacer” or “hinge” as used herein refers to the hydrophilic region which is between the antigen binding domain and the transmembrane domain. The CARs of the invention may comprise an extracellular spacer domain but is it also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge.

The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. The splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (the intracellular signaling domain or the activating endodomain) of the CAR is responsible for activation of at least one of the normal effector functions of the immune cell in which the CAR is expressed, if the respective CAR is an activating CAR (normally, a CAR as described herein refers to an activating CAR, otherwise it is indicated explicitly as an inhibitory CAR (iCAR)). “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRζ (CD3ζ), FcRgamma, FcRbeta, CD3gamma, CD3delta, CD3epsilon, CD5, CD22, CD79a, CD79b, and CD66d. Most prominent is sequence derived from CD3ζ.

The cytoplasmic domain of the CAR may be designed to comprise the CD3ζsignaling domain by itself or combined with any other desired cytoplasmic domain(s). The cytoplasmic domain of the CAR can comprise a CD3ζchain portion and a co-stimulatory signaling region (domain).

The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

As aforementioned either the extracellular part or the transmembrane domain or the cytoplasmic domain of a CAR may also comprise a heterodimerizing domain for the aim of splitting key signaling and antigen recognition modules of the CAR.

The CAR may be further modified to include on the level of the nucleic acid encoding the CAR one or more operative elements to eliminate CAR expressing immune cells by virtue of a suicide switch. The suicide switch can include, for example, an apoptosis inducing signaling cascade or a drug that induces cell death. In one embodiment, the nucleic acid expressing and encoding the CAR can be further modified to express an enzyme such thymidine kinase (TK) or cytosine deaminase (CD). The CAR may also be part of a gene expression system that allows controlled expression of the CAR in the immune cell. Such a gene expression system may be an inducible gene expression system and wherein when an induction agent is administered to a cell being transduced with said inducible gene expression system, the gene expression system is induced and said CAR is expressed on the surface of said transduced cell.

In some embodiments, the endodomain may contain a primary cytoplasmic signaling domains or a co-stimulatory region, but not both.

In some embodiment of the invention the CAR may be a “SUPRA” (split, universal, and programmable) CAR, where a “zipCAR” domain may link an intra-cellular costimulatory domain and an extracellular leucine zipper (WO2017/091546). This zipper may be targeted with a complementary zipper fused e.g. to an scFv region to render the SUPRA CAR T cell tumor specific. This approach would be particularly useful for generating universal CAR T cells for various tumors; adapter molecules could be designed for tumor specificity and would provide options for altering specificity post-adoptive transfer, key for situations of selection pressure and antigen escape.

The CARs of the present invention may be designed to comprise any portion or part of the above-mentioned domains as described herein in any order and/or combination resulting in a functional CAR, i.e. a CAR that mediated an immune effector response of the immune effector cell that expresses the CAR as disclosed herein.

The term “tagged polypeptide” as used herein refers to a polypeptide that has bound thereto directly or indirectly at least one additional component, i.e. the tag. The tagged polypeptide as used herein is able to bind an antigen expressed on a target cell. The polypeptide may be an antibody or antigen binding fragment thereof that binds to an antigen expressed on the surface of a target cell such as a tumor associated antigen on a cancer cell. The polypeptide of the tagged polypeptide alternatively may be a cytokine or a growth factor or another soluble polypeptide that is capable of binding to an antigen of a target cell.

The terms “adapter” or “adapter molecule” or “tagged polypeptide” as used herein may be used interchangeably.

The tag may be e.g. a hapten or dextran and the hapten or dextran may be bound by the antigen binding domain of the polypeptide, e.g. a CAR, comprising an antigen binding domain specific for the tag.

Haptens such as e.g. FITC, biotin, PE, streptavidin or dextran are small molecules that elicit an immune response only when attached to a large carrier such as a protein; the carrier may be one that also does not elicit an immune response by itself. Once the body has generated antibodies to a hapten-carrier adduct, the small-molecule hapten may also be able to bind to the antibody, but it will usually not initiate an immune response; usually only the hapten-carrier adduct can do this.

But the tag may also be a peptide sequence e.g. chemically or recombinantly coupled to the polypeptide part of the tagged polypeptide. The peptide may be selected from the group consisting of c-Myc-tag, Strep-Tag, Flag-Tag, and Polyhistidine-tag. The tag may also be streptavidin. The tag portion of the tagged polypeptide is only constrained by being a molecular that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv.

As used herein, a “T cell receptor” or “TCR” refers to the antigen-recognition molecules present on the surface of T-cells. During normal T-cell development, each of the four TCR genes, a, p y, 6 can rearrange leading to highly diverse TCR proteins.

The term “antibody” as used herein is used in the broadest sense to cover the various forms of antibody structures including but not being limited to monoclonal and polyclonal antibodies (including full length antibodies), multispecific antibodies (e.g. bispecific antibodies), antibody fragments, i.e. antigen binding fragments of an antibody, immunoadhesins and antibody-immunoadhesin chimeras, that specifically recognize (i.e. bind) an antigen. “Antigen binding fragments” comprise a portion of a full-length antibody, preferably the variable domain thereof, or at least the antigen binding site thereof (“an antigen binding fragment of an antibody”).

Examples of antigen binding fragments include Fab (fragment antigen binding), scFv (single chain fragment variable), single domain antibodies (nanobodies), diabodies, dsFv, Fab′, diabodies, single-chain antibody molecules, and multispecific antibodies formed from antibody fragments. The antibody or antibody fragment may be human, fully human, humanized, human engineered, non-human, and/or chimeric. The non-human antibody or antibody fragment may be humanized to reduce immunogenicity to humans, while retaining the specificity and affinity of the parental non-human antibody. Chimeric antibodies may refer to antibodies created through the joining of two or more antibody genes which originally encoded for separate antibodies.

The terms “having specificity for”, “specifically binds” or “specific for” with respect to an antigen-binding domain of an antibody, of a fragment thereof or of a CAR refer to an antigen-binding domain which recognizes and binds to a specific antigen, but does not substantially recognize or bind other molecules in a sample. An antigen-binding domain that binds specifically to an antigen from one species may bind also to that antigen from another species.

This cross-species reactivity is not contrary to the definition of that antigen-binding domain is specific. An antigen-binding domain that specifically binds to an antigen may bind also to different allelic forms of the antigen (allelic variants, splice variants, isoforms etc.). This cross reactivity is not contrary to the definition of that antigen-binding domain is specific.

As used herein, the term “antigen” is intended to include substances that bind to or evoke the production of one or more antibodies and may comprise, but is not limited to, proteins, peptides, polypeptides, oligopeptides, lipids, carbohydrates such as dextran, haptens and combinations thereof, for example a glycosylated protein or a glycolipid. The term “antigen” as used herein refers to a molecular entity that may be expressed e.g. on the surface of a target cell and that can be recognized by means of the adaptive immune system including but not restricted to antibodies or TCRs, or engineered molecules including but not restricted to endogenous or transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

The term “soluble antigen” as used herein refers to an antigen that is not immobilized on surfaces such as beads or cell membranes.

The terms “immune cell” or “immune effector cell” may be used interchangeably and refer to a cell that may be part of the immune system and executes a particular effector function such as alpha-beta T cells, NK cells, NKT cells, B cells, innate lymphoid cells (ILC), cytokine induced killer (CTK) cells, lymphokine activated killer (LAK) cells, gamma-delta T cells, regulatory T cells (Treg), monocytes or macrophages. Preferentially these immune cells are human immune cells. Preferred immune cells are cells with cytotoxic effector function such as alpha-beta T cells, NK cells, NKT cells, ILC, CIK cells, LAK cells or gamma-delta T cells.

Most preferred immune effector cells are T cells and NK cells. Tumor infiltrating lymphocytes (TILs) are T cells that have moved from the blood of a subject into a tumor. These TILs may be removed from a patient's tumor by methods well known in the art, e.g. enzymatic and mechanic tumor disruption followed by density centrifugation and/or cell marker specific enrichment. TILs are genetically engineered as disclosed herein, and then given back to the patient. “Effector function” means a specialized function of a cell, e.g. in a T cell an effector function may be cytolytic activity or helper activity including the secretion of cytokines.

Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, preferentially T cell-based or NK cell-based cytotoxic responses to attack cancer cells. T cells that have a natural or genetically engineered reactivity to a patient's cancer are generated in-vitro and then transferred back into the cancer patient. Then the immunotherapy is referred to as “CAR cell immunotherapy” or in case of use of T cells only as “CAR T cell therapy” or “CAR T cell immunotherapy”.

The term “treatment” as used herein means to reduce the frequency or severity of at least one sign or symptom of a disease.

The term “autologous” as used herein refers to any material derived from the same subject to who it is later re-introduced.

The term “allogeneic” as used herein refers to any material derived from a different subject of the same species as the subject to who the material is re-introduced.

The terms “therapeutically effective amount” or “therapeutically effective population” mean an amount of a cell population which provides a therapeutic benefit in a subject.

As used herein, the term “subject” refers to an animal. Preferentially, the subject is a mammal such as mouse, rat, cow, pig, goat, chicken dog, monkey or human. More preferentially, the subject is a human. The subject may be a subject suffering from a disease such as cancer (a patient) or from an autoimmune disease or from a allergic disease or from an infectious disease or from graft rejection.

The term “expression” as used herein is defined as the transcription of a particular nucleotide sequence into RNA and optionally subsequent translation of said RNA into a polypeptide sequence or a protein.

The terms “engineered cell” and “genetically modified cell” as used herein can be used interchangeably. The terms mean containing and/or expressing a foreign gene or nucleic acid sequence which in turn modifies the genotype or phenotype of the cell or its progeny. Especially, the terms refer to the fact that cells, preferentially T cells can be manipulated by recombinant methods well known in the art to express stably or transiently peptides or proteins which are not expressed in these cells in the natural state. For example, T cells, preferentially human T cells are engineered to express an artificial construct such as a chimeric antigen receptor on their cell surface.

The term “cancer” is known medically as a malignant neoplasm. Cancer is a broad group of diseases involving unregulated cell growth and includes all kinds of leukemia. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

The terms “nucleic acid”, “nucleic acid sequence” or “polynucleotide” as used interchangeably herein refer to polymers of nucleotides. Polynucleotides, which can be hydrolyzed into monomeric “nucleotides.” The monomeric nucleotides can be hydrolyzed into nucleosides. As used herein, the term “polynucleotides” encompasses, but is not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

The term “operably linked” refers to functional linkage between a regulatory sequence (e.g. a promoter) and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the terms “promoter” or “regulatory sequence” mean a nucleic acid sequence which is required for transcription of a gene product (a polynucleotide and/or a polypeptide/protein) operably linked to the promoter/regulatory sequence. In some instances, this sequence may be the core promoter sequence and in other instances, this sequence may also include an enhancer sequence and other regulatory elements which are required for transcription of the gene product. The promoter/regulatory sequence may, for example, be one which expresses the gene product in a tissue-specific manner.

The term “minimal promoter” as used herein refers to the smallest genetic element that is able to induce transcription of a gene located downstream of said minimal promoter. Eukaryotic promoters of protein-coding genes have one or more of three conserved sequences in this region (i.e. the TATA-box, initiator region, and downstream promoter element). A minimal promoter enables low basal leakiness in the absence of specific transcriptional activators and high expression when transcription activators are bound upstream of minimal promoter at their specific DNA binding sites. Alternative minimal promoters can be used, such as minimal TATA box promoter, minimal CMV promoter or minimal I1L-2 promoter.

The minimal promoter may be engineered/modified by the introduction of binding sites for specific transcription factors (e.g. required for the drug-inducible system).

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only in the presence or absence of certain conditions such as, for example, when an inducer (e.g. an induction signal, or an induction agent such as a drug, metal ions, alcohol, oxygen, etc.) is present in the cell. A drug-inducible promoter is a promoter that may be regulatable by the presence or absence of said drug that induces the promoter.

Constitutive promotors that are operatively linked to a transgene, e.g. the synthetic transcription factor as disclosed herein, may be for example EF-1 alpha promoter or any other constitutive promoter that drives constitutive expression in immune cells (such as MSCV, PGK-1, UBC, CMV, CAGG, SV40 or pan-hematopoietic promoter, such as vav).

The inducible promoter may comprise a minimal promoter (PMIN) such as E1b minimal promoter. Alternative minimal promoters can be used, such as minimal TATA box promoter, minimal CMV promoter or minimal I1L-2 promoter. In some embodiments, the minimal promoter may be optimized for a desired level or rate of transcription.

In a specific variant, the inducible promoter may be a drug-inducible promoter.

Such a system may comprise a nucleic acid comprising a promoter inducible by a drug, e.g. by a synthetic drug such as a metabolite of tamoxifen. By utilizing a drug-inducible promoter, a transgene expression may be turned on and off in order to avoid toxic side effects of the transgene and/or to allow the cells to rest during remission. Many of these systems use chimeric transcriptional regulators (e.g. synthetic transcription factors).

In one variant the inducible promoter may be inducible by a drug, i.e. a drug-inducible promoter. The drug is selected based on safety record, favorable pharmacokinetic profile, tissue distribution, a low partition coefficient between the extracellular space and cytosol, low immunogenicity, low toxicities, and/or high expression in lymphocytes. In some alternatives, the inducible promoter is activated by a transcriptional activator (e.g. a synthetic transcription factor) that interacts with a drug. The transcriptional activator is activated or able to bind to and activate the inducible promoter in the presence of the drug. A specific alternative of a drug is a drug that binds to an estrogen receptor ligand binding domain of a transcriptional activator. In some alternatives, the drug includes tamoxifen, its metabolites, analogs, and pharmaceutically acceptable salts and/or hydrates or solvates thereof.

The term “synthetic transcription factor” as used herein may comprise a DNA-binding domain, a ligand binding domain (a drug inducible domain or a drug binding domain) and an activation (effector) domain, that are linked and/or fused whereby the individual domains may be arranged in any order.

A DNA binding domain of a synthetic transcription factor may be a protein or a portion of a protein that specifically recognizes the DNA binding site of the drug-inducible promoter and mediates the binding of the synthetic transcription factor to this DNA sequence. Besides zinc finger proteins, TALE (transcription activator-like effector) and Cas9 (Clustered Regulatory Interspaced Short Palindromic Repeats -associated system) may be engineered to recognize a specific DNA sequence. Moreover, the DNA binding domain of naturally occurring transcription factors (e.g. POU homeodomain) may be employed.

Said DNA binding domain as disclosed herein may be a zinc-finger protein.

DNA binding sites (that are composed of responsive elements) of drug-inducible promoters are specific DNA sequences that are directly or indirectly (in case of Cas9) recognized by the DNA-binding domain of the synthetic transcription factor. E.g. each zinc finger domain specifically recognizes a DNA sequence of 3 bp (referred to as target site), thus a three-finger zinc finger protein can be designed to recognize a 9 bp sequence (referred to as recognition motif).

The Cys2His2 zinc finger (ZF) protein is the most common DNA-binding domain found in eukaryotes and represents the second most frequently encoded protein domain in the human genome with approximately 700 Cys2His2 ZF coding genes. Besides the classical ZF Cys2His2 protein, at least two more classes of zinc-coordinating DNA-binding proteins can be distinguished based on their structure and way of coordinating zinc. Cys2Cys2 (or Cys4) ZFs contain a conserved Zn-binding consensus of Cys-X2-Cys-X13-Cys-X2-Cys and can be found mainly in nuclear steroid or hormone receptors (e.g. estrogen receptor). A second class is characterized by the Cys6-zinc cluster occurring, for example, in metabolic regulators of fungi (e.g. yeast GAL4). Although the DNA-binding mechanism of all three classes of zinc-coordinating DNA-binding proteins is quite well understood, it is mainly Cys2His2 fingers that are used for the design of polydactyl ZFPs with novel and unique DNA specificities. The Cys2His2 ZF domain consists of approximately 30 amino acids and folds into a conserved left-handed Opa-secondary structure that is internally stabilized by hydrophobic interactions and the chelation of a zinc ion. The zinc atom is coordinated by the conserved cysteines within the 0-sheet hairpin and histidines within the α-helix. X-ray crystal structure analysis revealed that one ZF domain typically recognizes 3 bps (referred to as target site) in the major groove of double-stranded DNA. Amino acids at positions -1, 3 and 6 with respect to the start of the α-helix thereby contact the 3′, middle and 5′ nucleotide of a 3 bp subsite, respectively. Additionally, the α-helical position 2 mediates cross-strand interaction by recognizing a base on the reverse strand complementary to the nucleotide recognized by the α-helical position 6 of the preceding finger. This cross-strand interaction synergistically links fingers into an overlapping array.

Proteins containing Cys2His2 ZF domains usually exhibit varying tandem repeats of ZF domains. For instance, the transcription factor Zif268 has three ZF domains specifically recognizing a DNA sequence of 9 bps (referred to as recognition motif).

A ligand binding domain (or a drug-binding domain of a synthetic transcription factor) refers to a protein or a portion of a protein that binds to a drug (a ligand). Upon drug binding, the drug-binding domain enables the transition from an inactive to an active synthetic transcription factor. This transition may include the release of inactivation factors and/or the translocation of the synthetic transcription factor from the cytoplasm to the nucleus. Examples of drug binding domains are nuclear receptors, extracellular domains of receptors, antigen/substance binding proteins (also dimerizers) and/or active sites of enzymes.

The ligand-binding domain (LBD) may be a modified human estrogen receptor as disclosed herein. Said modified human estrogen receptor may comprise a modification that may result in the loss of responsiveness to the natural ligand, i.e. estrogen, but not to a synthetic ligand, i.e. the drug such a metabolite of tamoxifen such as 4-hydroxytamoxifen or endoxifen.

Said modified human estrogen receptor may comprise the amino acid substitutions G400V, M543A, L544A or G400V, L540A, M543A or G400V/L539A/L540A) as disclosed herein. Said modified human estrogen receptor, wherein said modified human estrogen receptor may comprise SEQ ID NO:10 or SEQ ID NO:11 or SEQ ID NO:12. Estrogen receptors (ERs) are a group of proteins found inside cells. They are receptors that are activated by the hormone estrogen (170-estradiol). Two classes of ER exist: nuclear estrogen receptors (ERα and ERβ), which are members of the nuclear receptor family of intracellular receptors, and membrane estrogen receptors (mERs) (GPER (GPR30), ER-X, and Gq-mER), which are mostly G protein-coupled receptor.

An activation domain of a synthetic transcription factor refers to a protein or a portion of a protein that autonomously facilitates the recruitment of the transcriptional machinery to initiate mRNA transcription. Examples of activation domains are VP16, VP64, fragments of NFkB p65, heat shock factor 1 and combinations thereof. The activation domain of the synthetic transcription factor as disclosed herein may be the p65 activation domain of the human transcription factor NFκB or a functional variant thereof.

NF-kappa-B (NKκB) is a pleiotropic transcription factor present in almost all cell types and is the endpoint of a series of signal transduction events that are initiated by a vast array of stimuli related to many biological processes such as inflammation, immunity, differentiation, cell growth, tumorigenesis and apoptosis. NF-kappa-B is a homo- or heterodimeric complex formed by the Rel-like domain-containing proteins RELA/p65, RELB, NFκB1/p105, NFκB1/p50, REL and NFκB2/p52. The heterodimeric RELA-NFκB1 complex appears to be most abundant one, wherein the p65 subunit is responsible for the strong transcription activating potential of NF-kappa-B Said drug-inducible promoter may be a hybrid promoter comprising a binding site for a DNA binding domain (DBD) and a minimal promoter that comprises a minimal promoter selected from the group consisting of E1b, TK, I1L2, CMV, SV40.

The term “drug-inducible (gene) expression system” or “drug-induced (gene) expression system” refers to the expression of an exogenous polynucleotide and/or polypeptide (a transgene), herein normally in a human (immune) cell. The inducible gene expression system may be activated in a cell having said inducible (gene) expression system, when a drug, e.g. a synthetic drug such as a metabolite of tamoxifen may be introduced to the cell. Said drug in the cell may bind to a synthetic transcription factor and subsequently may lead to the induction of the expression of the polypeptide and/or the transgene. Said drug may also be referred to as “inducing agent”.

In the presence of an induction agent, the inducible expression system drives expression of the exogenous polynucleotide and/or polypeptide. In an induced system, withdrawal of the induction agent may reduce and/or halt expression of the exogenous polynucleotide and/or polypeptide. Upon re-introduction of the induction agent, the system can then be re-induced and restarts the expression of the exogenous polynucleotide and/or polypeptide.

In some embodiments, an inducible (gene) expression system as disclosed herein may also provide tunable control of the expression of the polynucleotide and/or polypeptide. As used herein, the term “tunable control” refers to the ability to control the expression level of the polynucleotide and/or polypeptide as disclosed herein. For example, the level of induced expression of a transgene may depend on the amount of induction signal that is present. For example, the presence of a higher amount of induction agent, e.g. a synthetic drug may induce higher levels of expression of a transgene as compared to the presence of a lower amount of induction agent. As such, the inducible or tunable expression of a polynucleotide and/or polypeptide may be dose-dependent with respect to the amount of induction agent present.

Besides the inducer drug dose, in some embodiments, an inducible (gene) expression system as disclosed herein may also provide tunable control of the expression of the polynucleotide and/or polypeptide by the number of response elements for the synthetic transcription factor. As used herein, the term “tunable control” may refer to the ability to control the expression level of the polynucleotide and/or polypeptide as disclosed herein. For example, the level of induced expression of a polynucleotide and/or polypeptide may depend on the number of response elements in other words the number of binding sites for the synthetic transcription factor within the inducible promoter. For example, upon binding of five synthetic transcription factor molecules to an inducible promoter comprising five responsive elements a transcriptional output i.e. a higher level of expression of a polynucleotide and/or polypeptide is induced as compared to constructs comprising two responsive elements within the inducible promoter. As such, the inducible or tunable expression of a polynucleotide and/or polypeptide may be dependent from the number of responsive elements for the synthetic transcription factor.

A specific alternative of a drug is a drug that binds to an estrogen receptor ligand binding domain. In some alternatives, the drug includes tamoxifen, its metabolites, analogs, and pharmaceutically acceptable salts and/or hydrates or solvates thereof.

Tamoxifen, CAS RN 10540-29-1, is also known as 2-(4-((1Z)-1,2-diphenyl-1-butenyl)phenoxy)-N,N-dimethylethanamine, or (Z)-2-(para-(1,2-Diphenyl-1-butenyl) phenoxy) -N,N-dimethylamine (IUPAC), and has a molecular formula of C26H29NO, M.W. 371.52. Tamoxifen is a selective Estrogen Receptor Modulator with tissue-specific activities.

Tamoxifen acts as an anti-estrogen (inhibiting agent) agent in the mammary tissue, but as an estrogen (stimulating agent) in cholesterol metabolism, bone density, and cell proliferation in the endometrium. Tamoxifen is frequently administered orally as a pharmaceutically acceptable salt.

Metabolites of tamoxifen are e.g. Ndesmethyltamoxifen (RN 31750-48-8, M.W. 357.494) and 4-hydroxytamoxifen (4-OHT). Additional cytochrome P-450 metabolites are cis-4-hydroxytamoxifen (RN 174592, M.W. 387.52; Afimoxifene, E-isomer), and 4′-hydroxytamoxifen ((Z)-4-(1-(4-(2-(dimethylamino)ethoxy)phenyl)-1-phenylbut-1-en-2-yl)phenol).

Compounds with structural similarity to tamoxifen include, but are not limited to, cis-tamoxifen (RN 13002-65-8, M.W. 371.521), 4-methyltamoxifen (RN 73717-95-5, M.W. 385.548), N-desmethyltamoxifen (RN 31750-48-8, M.W. 357.494), (Z)-desethyl methyl tamoxifen (RN 15917-50-7, M.W. 357.494), (E)-desethyl methyl tamoxifen (RN 31750-45-5, M.W. 357.494), trans-4-hydoxytamoxifen (RN 68047-06-3, M.W. 387.52), Afimoxifene (RN 68392-35-8, M.W. 387.52, 4-hydroxytamoxifen), Afimoxifene, E-isomer (RN 174592-47-3, M.W. 387.52), 4-chlorotamoxifen (RN 77588-46-6, M.W. 405.966), 4-fluorotamoxifen (RN 73617-96-6, M.W. 389.511), Toremifene (RN 89778-26-7, M.W. 405.966), desethyl tamoxifen (RN 19957-51-8, M.W. 343.47), (E)-desethyl tamoxifen (RN 97151-10-5, M.W. 343.47), (Z)-desethyl tamoxifen (RN 97151-11-6, M.W. 343.47), Miproxifene (RN 129612-87-9, M.W. 429.6), 2-(p-(beta-ethylalpha-phenylstyryl)phenoxy)triethylamine (RN 749-86-0, M.W. 399.575), Droloxifene (RN 82413-20-5, M.W. 387.52), 4-iodo-tamoxifen (RN 116057-68-2, M.W. 497.413), dihydrotamoxifen (RN 109640-20-2, M.W. 373.537), (E)-N,N-dimethyl-2-(4-(1-(2-methylphenyl)-2-phenyl-1-butenyl)phenoxy)ethanamine (RN 97150-96-4, M.W. 385.548), or 4-hydroxytoremifene (RN 110503-62-3, M.W. 421.965); and/or pharmaceutically acceptable salts and/or hydrates or solvates thereof.

The N-terminus (also known as the amino-terminus, NH2-terminus, N-terminal end or amine-terminus) is the start of a protein or polypeptide referring to the free amine group (—NH2) located at the end of a polypeptide.

The C-terminus (also known as the carboxyl-terminus, carboxy-terminus, C-terminal tail, C-terminal end, or COOH-terminus) is the end of an amino acid chain (protein or polypeptide), terminated by a free carboxyl group (—COOH).

A kit may comprise a container with components within the container. Such containers may be e.g. boxes, bottles, vials, tubes, bags, pouches, blister packs, or other suitable container forms known in the art. Such containers may be made of plastic, glass, laminated paper, metal foil, or other materials suitable for holding components therein. The kit further may comprise written directions for using the components of the kits.

### Embodiments

In one embodiment of the invention an immune cell such as a T cell may comprise a system for tamoxifen or a derivative thereof such as 4-hydroxytamoxifen (4-OHT) inducible expression of a chimeric antigen receptor (CAR) specific for an antigen on the surface of a cancer cell, the system may comprise
a) a first nucleic acid sequence comprising a first promoter inducible by tamoxifen or a derivative thereof such as 4-OHT, wherein the first promoter may be operably linked to a polynucleotide encoding said CAR, wherein said first promotor may comprise a binding site for a DNA binding domain (DBD), wherein said binding site may comprise at least one responsive element that is recognized (bound) by said a DNA binding domain (DBD), and
b) a second nucleic acid sequence comprising a second promoter, wherein said second promoter may be a constitutive promoter, wherein the second promoter may be operably linked to a nucleic acid sequence encoding a synthetic transcription factor, wherein said synthetic transcription factor may comprise
i) an activation domain (AD), wherein said AD may comprise the p65 activation domain of the human transcription factor NFκB
ii) a DNA binding domain (DBD), wherein said DNA binding domain (DBD) may be the N1 zinc-finger protein (N1 ZFP)
iii) a ligand-binding domain (LBD), wherein said LBD may be a modified human estrogen receptor, wherein said modified human estrogen receptor may comprise the amino acid substitutions G400V, M543A, L544A as compared to the amino acid sequence of the wildtype human estrogen receptor, wherein said ligand-binding domain (LBD) is positioned at the C-terminus of said synthetic transcription factor.

Said immune cell may express said CAR upon contact with tamoxifen or a derivative thereof such as 4-OHT.

In one embodiment of the invention immune cells expressing inducible a CAR by tamoxifen or a derivative thereof such as 4-OHT as disclosed herein are for use in treatment of a disease associated with a target cell of a subject suffering from said disease, the disease may be e.g. cancer and the target cell a cancerous cell. Immune cells, e.g. T cells or NK cells of a subject may be isolated. The subject may e.g. suffer from said cancer or may be a healthy subject. These cells may be genetically modified in vitro to express the CAR upon contact with tamoxifen or a derivative thereof such as 4-OHT as disclosed herein. These engineered cells may be activated and expanded in vitro or in vivo to therapeutic effective amounts using methods known in the art. In a cellular therapy these engineered cells may be infused to a recipient in need thereof.

These cells may be a pharmaceutical composition (said cells plus pharmaceutical acceptable carrier). The infused cells may be e.g. able to kill (or at least stop growth of) cancerous cells in the recipient. The recipient may be the same subject from which the cells were obtained (autologous cell therapy) or may be from another subject of the same species (allogenic cell therapy).

The immune cells, preferentially T cells or NK cells engineered to inducibly express a CAR by tamoxifen or a derivative thereof such as 4-OHT as disclosed herein may be administered either alone, or as a pharmaceutical composition in combination with diluents and/or with other components such as IL-2 or other cytokines or cell populations. Briefly, pharmaceutical compositions of the present invention may comprise a cell population of genetically modified cells (a plurality of immune cells) as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Preferentially, the compositions of the present invention may be formulated for intravenous administration. The administration of cell compositions to the subject may be carried out in any convenient manner known in the art.

Pharmaceutical compositions of the present invention may be administered in a manner appropriate to the disease to be treated. Appropriate dosages may be determined by clinical trials. But the quantity and frequency of administration will also be determined and influenced by such factors as the condition of the patient, and the type and severity of the patient's disease.

A pharmaceutical composition comprising the immune cells, preferentially T cells or NK cells expressing the inducible expression system as disclosed herein may be administered at a dosage of 10⁴ to 10⁹ cells/kg body weight, preferably 10⁵ to 10⁶ cells/kg body weight. The cell compositions may also be administered several times at these dosages. The compositions of cells may be injected e.g. directly into a tumor, lymph node, or site of infection.

In addition to the pharmaceutical composition comprising said genetically engineered immune cells, the inducing agent tamoxifen or a derivative thereof such as 4-OHT may be administered to said subject intended to be treated. Said inducing agent may be administered to said subject simultaneously with the administration of said immune cells, or may be administered subsequently, e.g. one day or one week later. Standard doses of tamoxifen range from 20-40 mg daily and may be given for up to 5 years. Peak plasma concentrations of tamoxifen may occur between approximately 5 and 7 h following a single oral dose of 20 mg with an average concentration of 40 ng/mL. Under steady-state conditions, obtained after 80 days of daily dosing, tamoxifen may reach plasma levels ranging from 71-183 ng/mL. Tamoxifen is extensively metabolized predominantly by CYP enzymes in the liver. Metabolism thereby follows two pathways, 4-hydroxylation and N-demethylation, resulting in 4-OHT and N-desmethyltamoxifen (NDM-tamoxifen), respectively. Finally, both primary metabolites are transformed into the secondary metabolite endoxifen.

A daily oral dosage of 4 mg of endoxifen is supposed to result in serum concentrations similar to those found in rapid metabolizers with standard tamoxifen treatment (20 mg/day).

### EXAMPLES

### Example 1: Generation of Drug-Inducible CAR T Cells

### 1.] Construct Design

Inducible T cells constitutively express a synthetic transcription factor via the PGK promoter. The synthetic transcription factor is composed of a 3-finger zinc finger protein, referred to as N1, the ligand binding domain of an human estrogen receptor and an activation domain derived from NFκB p65 (in different orientations of the individual domains). The sequence of the synthetic transcription factor is linked via a furin P2A site to LNGFR, which was used as transduction marker. The inducible gene expression cassette also comprises the sequence for the anti-CD20 CAR whereby the transcription of the anti-CD20 CAR is regulated by the inducible promoter. The 2nd generation anti-CD20 CAR incorporates a leader sequence from huGM-CSFR, an anti-CD20 scFv based on Leu-16, CD8 hinge, and transmembrane domain, as well as the cytoplasmic domains of 4-1BB and CD3ζ. The inducible promoter is composed of the binding site for the N1 zinc finger (five responsive elements) linked to an E1b minimal promoter.

### 1.2 Generation of LV Particles and Titration

Lentiviral vector particles were manufactured via transient transfection of HEK-293 T cells. The lentiviral vector particles were pseudotyped with VSV-G. For transfection HEK-293T cells were seeded in T175 culture flasks in DMEM (Biowest) supplemented with 2 mM L-Glutamine (Lonza) and 10% FCS (Biochrom) 3 days prior to transfection. At the day of transfection the culture medium was removed and replaced by DMEM (Biowest) supplemented with 2 mM L-Glutamine (Lonza). The cells were transfected with a three plasmid system encoding for VSV-G, gag/pol/rev and the psi positive transfer vector (anti-tag CAR or inducible cassette). After 48 h the supernatant was collected and centrifuged for 10 min at 1000 rpm to remove cellular debris. In addition, the supernatant was filtrated trough a 0.45 μm filter. The pellet was re-suspended in ice cold PBS and stored at−80° C.

A functional titer of VSV-G pseudotyped lentiviral vector particles was determined via titration on Sup-T1 cells. 2E5 cells were seeded in 100 μL RPMI (Biowest) supplemented with 2 mM L-Glutamine (Lonza) in 96 well round bottom plates. For transduction 100 μL of serial diluted lentiviral vector particles were added to the seeded cells. 90 μL RPMI (Biowest) supplemented with 2 mM L-Glutamine (Lonza) and 10% FCS (Biochrom) was added after 24 h. The frequency of transduced cells was quantified after 96 h by flow cytometry using a LNGFR APC conjugate (Miltenyi Biotec). Based on the frequency of LNGFR positive cells, the number of seeded cells and the volume of lentiviral particle used for transduction, the titer was calculated. The titer was expressed in transducing units per mL.

1.3 Transduction, Cultivation and Analysis of Inducible CAR T Cells Inducible CAR T cells were manufactured using primary T cells from healthy donors. T cells were isolated from PBMC with the PAN T cell isolation Kit (Miltenyi Biotec) according to the manufactures protocol. Prior to transduction 2E6 T cells were seeded in a 24 well plate with 2 mL TexMACS medium (Miltenyi Biotec) supplemented with IL-7 (Miltenyi Biotec), IL-15 (Miltenyi Biotec) and TransAct (Miltenyi Biotec). After 24 h T cells were transduced with an MOI of 5 by adding the corresponding volume of lentiviral vector particles. On day 3 post activation the culture medium was removed and replaced by TexMACS medium (Miltenyi Biotec) supplemented with IL-7 (Miltenyi Biotec) and IL-15 (Miltenyi Biotec). Frequency of inducible CAR T cells was indirectly analyzed on day 6 after transduction via flow cytometric determination of LNGFR expression using a anti-LNGFR-APC conjugate (Miltenyi Biotec). Transduced T cells were enriched for LNGFR positive cells on day 7 post transduction using MACSelect LNGFR MicroBeads (Miltenyi Biotec). Enrichment procedure was done according to suppliers protocol. Transduced T cells were used for functional assays on day 13 after activation.

### Example 2: Induction of Anti-CD20 CAR Expression in Primary T Cells

Lentiviral particles encoding for the different drug-inducible cassettes were manufactured as described in example 1.2. Drug-inducible T cells were generated as described in example 1.3. 1E4 LNGFR positive drug-inducible T cells were seeded in 100 μL TexMACS medium (Miltenyi Biotec) supplemented with IL-7 (Miltenyi Biotec) and IL-15 (Miltenyi Biotec) in a 96-round bottom well plate. Anti-CD20 CAR expression was induced by the addition of different concentrations of 4-OHT (0-250 nM, Sigma-Aldrich) in 100 μL μL TexMACS medium (Miltenyi Biotec) supplemented with IL-7 (Miltenyi Biotec) and IL-15 (Miltenyi Biotec) to each well. Untransduced T cells constitute the negative control. T cells that 10 constitutively express an anti-CD20 CAR (“direct CAR”) represent the positive control. Cells were incubated at 37° C. and 5% CO₂. T cells were analyzed 40h post induction for anti-CD20 CAR expression by flow cytometry using anti-CD20 CAR detection reagent-PE. Therefore, T cells were pelleted at 300 g for 5 min and stained with 50 μL staining mixture composed of the anti-CD20 CAR detection reagent-PE and anti-LNGFR-APC diluted in CliniMACS buffer (Miltenyi Biotec) supplemented with 0.5% BSA (Miltenyi Biotec) (referred to as PEB).

Staining was performed for 10 min at 4° C. followed by two successive washing steps with 200 L PEB (300 g, 5 min). Cells were resuspended in 100 L PEB for subsequent flow cytometric analysis. To exclude dead cells, Propidium iodide (PI; Miltenyi Biotec) was added to the stained cells directly before sample acquisition at the MACSQuant® Analyzer 10 (Miltenyi Biotec). Frequency of anti-CD20 CAR+inducible T cells (left graph) and mean fluorescence intensity (MFI) of anti-CD20 CAR expression (right graph) was determined and normalized to the expression of the transduction marker LNGFR.

Induction of anti-CD20 CAR expression by inducible T cells (both constructs) is strictly dependent on the presence of the inducer drug 4-OHT with higher drug doses resulting in greater induction ratios. Concurrently, surface expression (represented as MFI) steadily increased with the drug dose.

### Example 3: Cytotoxic Activity of Inducible Anti-CD20 CAR T Cells in Co-Cultures with GFP+CD20⁺ Mel526

To assess the tumor cell lysis mediated by inducible CAR T cells in real-time, a live cell imaging-based killing assay using the GFP+CD20+tumor cell line 526-Mel was performed. Therefore, 1E4 GFP+CD20+Mel526 cells were seeded in 50 μL TexMACS medium (Miltenyi Biotec) in a 96-well flat-bottom plate. Inducible CAR T cells were subsequently added at an E:T ratio of 1:1 in a volume of 100 uL TexMACS medium (Miltenyi Biotec). T cell numbers were adjusted to LNGFR expression implicating equal transduced and total T cell numbers in each well. The number of untransduced T cells was adjusted to total cell numbers. Anti-CD20 CAR expression was induced by the addition of 100 nM 4-OHT (Sigma-Aldrich) in 50 μL TexMACS (Miltenyi Biotec) at the start of the assay. The plate was centrifuged at 300 g for 1 min and transferred into the IncuCyte® device. The IncuCyte® S3 device automatically acquires images of the labeled tumor cells periodically overtime and correlates the fluorescence signal to tumor cell growth.

Following an initial latency period of approximately 20 h, inducible anti-CD20 CAR T cells efficiently lysed GFP+CD20+526-Mel cells in the presence of 4-OHT. Elimination rates were comparable between the two inducible constructs and slightly delayed compared to the conventional constitutively expressed anti-CD20 CAR (“direct CAR”). Tumor-specific cytotoxicity of inducible CAR T cells was strictly dependent on the presence of 4-OHT characterized by the absence of basal lysis in the non-induced state. In the untransduced control group, tumor cells remained in a steady growth phase throughout the experimental period.

### Example 4: Cytokine Secretion of Inducible Anti-CD20 CAR T Cells in Co-Cultures with GFP⁺

CD20+Mel526. The capacity of inducible anti-CD20 CAR T cells to secrete effector cytokines in co-cultures with tumor cells was analyzed using human MACS Plex 12 cytokine kit (Miltenyi Biotec). Therefore, 1E4 GFP+CD20+Mel526 cells were seeded in 50 μL TexMACS medium (Miltenyi Biotec) in a 96-well flat-bottom plate. Inducible CAR T cells were subsequently added at an E:T ratio of 1:1 in a volume of 100 μL TexMACS medium (Miltenyi Biotec). T cell numbers were adjusted to LNGFR expression implicating equal transduced and total T cell numbers in each well. The number of untransduced T cells was adjusted to total cell numbers. Anti-CD20 CAR expression was induced by the addition of 100 nM 4-OHT (Sigma-Aldrich) in 50 μL TexMACS (Miltenyi Biotec) at the start of the assay. The plate was centrifuged at 300 g for 1 min and incubated at 37° C., 5% CO₂ for 40h. Then, 100 μl co-culture supernatant was transferred into a new 96-well plate and cytokine levels were analyzed using human MACS Plex 12 cytokine kit (Miltenyi Biotec) according to manufacturer's instructions.

The secretion of human cytokines (hIFN-7, left panel and hIL-2, right panel) by inducible anti-CD20 CAR T cells (both constructs) upon antigen engagement was strictly dependent on the presence of 4-OHT. In the non-induced state, cytokine levels in the supernatant were comparable to the negative, untransduced control. Induction of inducible anti-CD20 CAR T cells (both constructs) resulted in higher cytokine levels compared to the constitutively expressed CAR under the control of the PGK promoter.

### Example 5: Cytotoxic Activity of Inducible Anti-CD20 CAR T Cells In Vivo

To study the in vivo functionality of inducible CAR T cells, the immunodeficient mouse strain NOD.Cg-PrkdcscidIL2rgtml Wjl/SzJ (NSG™) was selected. For tumor establishment 4×10⁵ mouse-adapted Raji^{ffluc} cells were injected i.v. in 100 uL PBS into the tail vein of NSG™ mice.

Tumors were engrafted for 7 days and tumor growth was monitored regularly by bioluminescent imaging and expressed in photon flux [photons/see] over time for individual mice. Prior to T cell injection, mice were randomized according to the BLI signal measured on day 0 for equally distributed tumor flux among the treatment groups. Mice were treated with untransduced, inducible or conventional constitutively expressed (“direct”) anti-CD20 CAR T cells. T cell numbers were adjusted to 3×10⁶ LNGFR⁺ T cells ensuring equal transduced and total T cell numbers per mouse. The number of untransduced T cells was adjusted to the total cell number injected in the CAR T cell groups. T cells were injected i.v. in 100 L PBS into the tail vein of NSG™ mice. Tumor only control groups (untreated) received i.v. injections of 100 L PBS. Tamoxifen (Sigma-Aldrich) was administered daily at a dose of 1 mg by i.p. injections. Therefore, tamoxifen was dispersed in 50 L peanut oil (Sigma-Aldrich) Control groups received vehicle injections only. Dosing was started on day 0.

The rapid tumor outgrowth within the inducible anti-CD20 CAR cohort receiving vehicle injections was comparable to the untreated control group demonstrating the absence of background anti-tumor efficacy in the non-induced state. In the presence of tamoxifen, inducible anti-CD20 CAR T cells with the synthetic transcription factor orientation N1-p65-ER showed profound Raji cell lysis in 4 out of 5 mice. However, elimination rates were slower compared to the conventional control CAR. The second inducible anti-CD20 CAR T cell cohort with the synthetic transcription factor orientation N1-ER-p65 reduced tumor growth in 2 out of 4 mice in the presence of tamoxifen.

### Example 6: Positioning of the Activation Domain within the Synthetic Transcription Factor Determines Background Expression in the Non-Induced State and the Transcriptional Output

T cells were transduced with synthetic transcription factors comprising a zinc finger protein (N1), the ligand binding domain of the estrogen receptor (ER), and an activation domain (p65) in distinct arrangements as described in example 1.2 and 1.3. Inducible anti-CD20 CAR T cells comprising the N1-ER-VP64 as synthetic transcription factor served as control. The assay was set up and carried out as described in example 3. Frequency of anti-CD20 CAR⁺ inducible T cells (left graph) and mean fluorescence intensity (MFI) of anti-CD20 CAR expression (right graph) was determined by flow cytometric analysis upon staining of cells with anti-CD20 CAR detection reagent-PE and anti-LNGFR-APC and normalized to the expression of the transduction marker LNGFR. No basal activity in the non-induced state was detected for inducible CAR T cells comprising synthetic transcription factor constructs with the ER in C-terminal position. However, N-terminal positioning of the ER within the synthetic transcription factor resulted in background expression in the absence of the inducer drug. Induction ratios increased with the inducer dose added to the T cell culture. Concurrently, surface expression (represented as MFI) steadily increased with the drug dose. Maximal anti-CD20 CAR surface expression upon addition of 250 nM 4-OHT was higher for synthetic transcription factor constructs with p⁶⁵ (288-548) as activation domain comprising the ER in N-terminal position compared to C-terminal position. Highest anti-CD20 CAR expression was detected for the control construct with the synthetic transcription factor N1-ER-VP64.

### Example 7: The Selection of the Activation Domain within the Synthetic Transcription Factor Determines Background Expression in the Non-Induced State and the Transcriptional Output

T cells were transduced with synthetic transcription factors comprising a zinc finger protein (N1), the ligand binding domain of the human estrogen receptor (ER), and distinct activation domains (either NFκB p65 (aa361-551), NFκB p65 (aa288-548) or a fusion protein of NFκB p65 and HSF1 (heat shock factor 1)) as described in example 1.2 and 1.3. Inducible anti-CD20 CAR T cells comprising the N1-ER-VP64 as synthetic transcription factor served as control. The assay was set up and carried out as described in example 3. Frequency of anti-CD20 CAR⁺ inducible T cells (left graph) and mean fluorescence intensity (MFI) of anti-CD20 CAR expression (right graph) was determined by flow cytometric analysis upon staining of cells with anti-CD20 CAR detection reagent-PE and anti-LNGFR-APC and normalized to the expression of the transduction marker LNGFR.

Induction of anti-CD20 CAR expression by inducible T cells was strictly dependent on the presence of the inducer drug if the synthetic transcription factor was composed of N1-p65(361-551)-ER, N1-p65(288-548)-ER or N1-ER-VP64. For those constructs, addition of higher drug doses to the T cell culture resulted in greater induction ratios. Concurrently, surface expression (represented as MFI) steadily increased with the drug dose. The maximal anti-CD20 CAR surface expression differed between constructs with N1-ER-VP64>N1-p65(361-551)-ER>N1-p65(288-548)-ER showing increasing levels. Interestingly, inducible T cells comprising a hybrid activation domain composed of p65 and HSF1 within the synthetic transcription factor arrangement N1-AD-ER showed basal activity in the non-induced state.

Sequences:
(recognition sequence; amino acid sequence):
SEQ ID NO: 1
SQSSNLVR
(recognition sequence; amino acid sequence):
SEQ ID NO: 2
SQSSSLVR
(recognition sequence; amino acid sequence):
SEQ ID NO: 3
SRSDKLVR
(consensus zinc-finger-framework sequence
derived from native and mutant versions
of Sp1 zinc fingers; X = any natural amino
acid):
SEQ ID NO: 4
MAQAALEPKEKPYACPECGKSFXXXXXXXXHQRTHTGEKPYKCPECGKSF
XXXXXXXXHQRTHTGEKPYKCPECGKSFXXXXXXXXHQRTHTGKKTSGQA
G
(N1 zinc-finger protein; amino acid
sequence):
SEQ ID NO: 5
MAQAALEPKEKPYACPECGKSFSQSSNLVRHQRTHTGEKPYKCPECGKSF
SQSSSLVRHQRTHTGEKPYKCPECGKSFSRSDKLVRHQRTHTGKKTSGQA
G
(responsive element for N1; DNA sequence):
SEQ ID NO: 6
ggggtagaaaaaggggtagaa
(Recognition motif for N1; DNA sequence,
n = a, c, g or t):
SEQ ID NO: 7
ggggtagaan
(p65 activation domain of the human
transcription factor NFKB; amino acid
sequence):
SEQ ID NO: 8
MDELFPLIFPAEPAQASGPYVEIIEQPKQRGMRFRYKCEGRSAGSIPGER
STDTTKTHPTIKINGYTGPGTVRISLVTKDPPHRPHPHELVGKDCRDGFY
EAELCPDRCIHSFQNLGIQCVKKRDLEQAISQRIQTNNNPFQVPIEEQRG
DYDLNAVRLCFQVTVRDPSGRPLRLPPVLSHPIFDNRAPNTAELKICRVN
RNSGSCLGGDEIFLLCDKVQKEDIEVYFTGPGWEARGSFSQADVHRQVAI
VFRTPPYADPSLQAPVRVSMQLRRPSDRELSEPMEFQYLPDTDDRHRIEE
KRKRTYETFKSIMKKSPFSGPTDPRPPPRRIAVPSRSSASVPKPAPQPYP
FTSSLSTINYDEFPTMVFPSGQISQASALAPAPPQVLPQAPAPAPAPAMV
SALAQAPAPVPVLAPGPPQAVAPPAPKPTQAGEGTLSEALLQLQFDDEDL
GALLGNSTDPAVFTDLASVDNSEFQQLLNQGIPVAPHTTEPMLMEYPEAI
TRLVTGAQRPPDPAPAPLGAPGLPNGLLSGDEDFSSIADMDFSALLSQIS
S
(human ER aa282 to aa595; amino acid
sequence):
SEQ ID NO: 9
SAGDMRAANLWPSPLMIKRSKKNSLALSLTADQMVSALLDAEPPILYSEY
DPTRPFSEASMMGLLTNLADRELVHMINWAKRVPGFVDLTLHDQVHLLEC
AWLEILMIGLVWRSMEHPGKLLFAPNLLLDRNQGKCVEGMVEIFDMLLAT
SSRFRMMNLQGEEFVCLKSIILLNSGVYTFLSSTLKSLEEKDHIHRVLDK
ITDTLIHLMAKAGLTLQQQHQRLAQLLLILSHIRHMSNKGMEHLYSMKCK
NVVPLYDLLLEMLDAHRLHAPTSRGGASVEETDQSHLATAGSTSSHSLQK
YYITGEAEGFPATV
(human ER(G400V, L540A, M543A); amino
acid sequence):
SEQ ID NO: 10
MSAGDMRAANLWPSPLMIKRSKKNSLALSLTADQMVSALLDAEPPILYSE
YDPTRPFSEASMMGLLTNLADRELVHMINWAKRVPGFVDLTLHDQVHLLE
CAWLEILMIGLVWRSMEHPVKLLFAPNLLLDRNQGKCVEGMVEIFDMLLA
TSSRFRMMNLQGEEFVCLKSIILLNSGVYTFLSSTLKSLEEKDHIHRVLD
KITDTLIHLMAKAGLTLQQQHQRLAQLLLILSHIRHMSNKGMEHLYSMKC
KNVVPLYDLALEALDAHRLHAPTSRGGASVEETDQSHLATAGSTSSHSLQ
KYYITGEAEGFPATV
(human ER(G400V, M543A, L544A); amino
acid sequence):
SEQ ID NO: 11
MSAGDMRAANLWPSPLMIKRSKKNSLALSLTADQMVSALLDAEPPILYSE
YDPTRPFSEASMMGLLTNLADRELVHMINWAKRVPGFVDLTLHDQVHLLE
CAWLEILMIGLVWRSMEHPVKLLFAPNLLLDRNQGKCVEGMVEIFDMLLA
TSSRFRMMNLQGEEFVCLKSIILLNSGVYTFLSSTLKSLEEKDHIHRVLD
KITDTLIHLMAKAGLTLQQQHQRLAQLLLILSHIRHMSNKGMEHLYSMKC
KNVVPLYDLLLEAADAHRLHAPTSRGGASVEETDQSHLATAGSTSSHSLQ
KYYITGEAEGFPATV
(human ER(G400V/L539A/L540A); amino
acid sequence):
SEQ ID NO: 12
MSAGDMRAANLWPSPLMIKRSKKNSLALSLTADQMVSALLDAEPPILYSE
YDPTRPFSEASMMGLLTNLADRELVHMINWAKRVPGFVDLTLHDQVHLLE
CAWLEILMIGLVWRSMEHPVKLLFAPNLLLDRNQGKCVEGMVEIFDMLLA
TSSRFRMMNLQGEEFVCLKSIILLNSGVYTFLSSTLKSLEEKDHIHRVLD
KITDTLIHLMAKAGLTLQQQHQRLAQLLLILSHIRHMSNKGMEHLYSMKC
KNVVPLYDAALEMLDAHRLHAPTSRGGASVEETDQSHLATAGSTSSHSLQ
KYYITGEAEGFPATV

1. A system for drug-inducible expression of a polynucleotide comprising
a) a first nucleic acid sequence comprising a first promoter inducible by said drug, wherein the first promoter is operably linked to said polynucleotide, wherein said first promotor comprises a binding site for a DNA binding domain, wherein said binding site comprises at least one responsive element that is recognized by said DNA binding domain (DBD), and
b) a second nucleic acid sequence comprising a second promoter, wherein the second promoter is operably linked to a nucleic acid sequence encoding a synthetic transcription factor, wherein said synthetic transcription factor comprises
i) an activation domain (AD), wherein said AD comprises the p65 activation domain of the human transcription factor NFκB,
ii) said DNA binding domain (DBD), wherein said DBD comprises or consists of 3 zinc finger domains
iii) a ligand-binding domain (LBD), wherein said LBD is a modified human estrogen receptor which is able to bind said drug, and wherein said ligand-binding domain (LBD) is positioned at the C-terminus of said synthetic transcription factor,
c) said drug, wherein said drug is tamoxifen or a metabolite of tamoxifen.

2. The system according to claim 1, wherein said synthetic transcription factor comprises from N-terminus to C-terminus said AD, said DBD, and said LBD or wherein said synthetic transcription factor comprises from N-terminus to C terminus said DBD, said AD and said LBD.

3. The system according to claim 1, wherein said DNA binding domain (DBD) is the N1 zinc finger protein (N1).

4. The system according to claim 1, wherein said modified human estrogen receptor comprises SEQ ID NO:10 or SEQ ID NO:11 or SEQ ID NO:12.

5. The system according to claim 1, wherein said metabolite of tamoxifen is 4-hydroxytamoxifen (4-OHT) or endoxifen.

6. The system according to claim 1, wherein said polynucleotide encodes a therapeutically active protein.

7. The system according to claim 1, wherein said polynucleotide encodes a chimeric antigen receptor (CAR).

8. The system according to claim 1, wherein said first nucleic acid sequence and said second nucleic acid sequence are on one nucleic acid sequence of a vector.

9. A composition comprising
A) a human immune cell comprising
a) a first nucleic acid sequence comprising a first promoter inducible by a drug, wherein the first promoter is operably linked to a polynucleotide, wherein said first promotor comprises a binding site for a DNA-binding domain, wherein said binding site comprises at least one responsive element that is recognized by the DNA-binding domain, and
b) a second nucleic acid sequence comprising a second promoter, wherein the second promoter is operably linked to a nucleic acid sequence encoding a synthetic transcription factor, wherein said synthetic transcription factor comprises
i) an activation domain (AD), wherein said AD comprises the p65 activation domain of the human transcription factor NFκB,
ii) said DNA binding domain (DBD), wherein said DBD comprises or consists of 3 zinc finger domains
iii) a ligand-binding domain (LBD), wherein said LBD is a modified human estrogen receptor which is able to bind said drug, and wherein said ligand-binding domain (LBD) is positioned at the C-terminus of said synthetic transcription factor,
B) said drug, wherein said drug is tamoxifen or a metabolite of tamoxifen.

10. A composition according to claim 9, wherein said human immune cell is a T cell or NK cell, and wherein said polynucleotide encodes a chimeric antigen receptor.

11. A composition for use in treatment of a disease comprising
A) a human immune cell comprising
a) a first nucleic acid sequence comprising a first promoter inducible by drug, wherein the first promoter is operably linked to a polynucleotide, wherein said first promotor comprises a binding site for a DNA-binding domain (DBD), wherein said binding site comprises at least one responsive element that is recognized by the DNA-binding domain, and
b) a second nucleic acid sequence comprising a second promoter, wherein the second promoter is operably linked to a nucleic acid sequence encoding a synthetic transcription factor, wherein said synthetic transcription factor comprises
i) an activation domain (AD), wherein said AD comprises the p65 activation domain of the human transcription factor NFκB,
ii) said DNA binding domain (DBD), wherein said DBD comprises or consists of 3 zinc finger domains
iii) a ligand-binding domain (LBD), wherein said LBD is a mutated human estrogen receptor which is able to bind said drug, and wherein said ligand-binding domain (LBD) is positioned at the C-terminus of said synthetic transcription factor,
B) said drug, wherein said drug is tamoxifen or a metabolite of tamoxifen.

12. The composition according to claim 11, wherein the disease is cancer, wherein the human immune cell is a T cell or NK cell, and wherein said polynucleotide encodes a chimeric antigen receptor specific for a tumor associated antigen (TAA) or tumor specific antigen (TSA) expressed on a cancerous cell of said cancer, and wherein said drug is 4-hydroxytamoxifen (4-OHT) or endoxifen.


SEQUENCE LISTING (12 sequences)

SEQ ID NO: 1 - protein, 8 aa, synthetic construct
SQSSNLVR

SEQ ID NO: 2 - protein, 8 aa, synthetic construct
SQSSSLVR

SEQ ID NO: 3 - protein, 8 aa, synthetic construct
SRSDKLVR

SEQ ID NO: 4 - protein, 101 aa, synthetic construct
MAQAALEPKEKPYACPECGKSFXXXXXXXXHQRTHTGEKPYKCPECGKSFXXXXXXXXHQRTHTGEKPYKCPECGKSFXXXXXXXXHQRTHTGKKTSGQAG

SEQ ID NO: 5 - protein, 101 aa, synthetic construct
MAQAALEPKEKPYACPECGKSFSQSSNLVRHQRTHTGEKPYKCPECGKSFSQSSSLVRHQRTHTGEKPYKCPECGKSFSRSDKLVRHQRTHTGKKTSGQAG

SEQ ID NO: 6 - DNA, 21 bp, synthetic construct
  source 1..21: mol_type = other DNA
ggggtagaaaaaggggtagaa

SEQ ID NO: 7 - skipped

SEQ ID NO: 8 - protein, 551 aa, synthetic construct
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQVPIEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 9 - protein, 314 aa, synthetic construct
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 10 - protein, 315 aa, synthetic construct
MSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLALEALDAHRLHAPTSRGGASVEETDQSHLATAGSTSSHSLQKYYITGEAEGFPATV

SEQ ID NO: 11 - protein, 315 aa, synthetic construct
MSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPATV

SEQ ID NO: 12 - protein, 315 aa, synthetic construct
MSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDAALEMLDAHRLHAPTSRGGASVEETDQSHLATAGSTSSHSLQKYYITGEAEGFPATV